(19) United States
(12) Patent Application Publication
(10) Pub. No.: US 2025/0032531 A1
(43) Pub. Date: Jan. 30, 2025

(54) TOPICALLY APPLICABLE PREPARATION FOR ENHANCING THE CONDITION OF SKIN

(71) Applicant: BEIERSDORF AG, Hamburg (DE)

(72) Inventors: Sascha BAUMANN, Grossenrade (DE); Christoph ERNST, Hamburg (DE); Andreas FIRYN, Schwarzenbek (DE); Thomas BLATT, Wedel (DE); Denise KUSCHEL, Hamburg (DE); Michael KUHN, Apensen (DE); Gerlinde PAPPA, Prisdorf (DE)

(21) Appl. No.: 18/706,811

(22) PCT Filed: Dec. 7, 2022
(86) PCT No.: PCT/EP2022/084692

§ 371 Date: May 2, 2024

(30) Foreign Application Priority Data
Dec. 9, 2021 (DE) 10 2021 214 021.1
Mar. 15, 2022 (DE) 10 2022 202 547.4

(51) Int. Cl. A61K 31/728; A61K 9/00; A61K 31/12; A61K 31/122; A61K 31/426; A61K 31/60; A61K 36/484; A61K 47/02; A61K 47/12; A61P 17/00

(52) U.S. Cl. CPC A61K 31/728; A61K 9/0014; A61K 31/12; A61K 31/122; A61K 31/426; A61K 31/60; A61K 36/484; A61K 47/02; A61K 47/12; A61P 17/00

(57) ABSTRACT

A topically applicable preparation, particularly a cosmetic or dermatological preparation, comprising a substance combination of water, hyaluronic acid and/or hyaluronate salt, one or more fruit acids, one or more skin moisturizers and one or more alcohols can be successfully used for skin care, especially for preventing and treating the damage caused by atopic dermatitis of the skin and caring for such skin, without containing lipids and emollients that are typically included.

The present invention relates to topically applicable preparations, in particular cosmetic or dermatological preparations, comprising a substance combination of hyaluronic acid and/or hyaluronic acid salt, one or more fruit acids, one or more skin moisturizing agents and one or more alcohols. The preparation does not comprise emollients and lipids. In particular, the present invention relates to the use of the preparations for skin care, in particular for care, prophylaxis and treatment of skin damaged by atopic dermatitis.

The cause of a large number of skin diseases, in particular atopic skin diseases, has not been explained in detail. Common to these diseases are inflammatory reactions in the dermis and in the dermal-epithelial transition zone. Considerable shifts in the normal hyaluronic acid content of the dermis and epidermis are known to occur in these diseases. To treat such diseases, various measures are currently used, for example administration of oily ointments, creams or lotions differing in active-ingredient additive. However, such inflammatory diseases are most effectively treated with corticoid-containing preparations for external (topical) use. The well-known local and systemic adverse effects of corticoids (derivatives of the endogenous steroid hormone cortisol) are accepted. Chronic use of topical corticoid preparations generally results in long-term consequences, such as skin atrophy.

From the field of preparations for oral use, WO 2008/009956 A1 discloses mouthwash solutions and sprays which contain hyaluronic acid and fruit acids for taste.

CN 111671708 discloses a preparation comprising a base composition comprising ethanol, glycerol, water, citric acid and 0.1-0.3% allantoin, an emollient, and a hyaluronic acid composition.

US 2014/0088040 A1 discloses a cosmetic preparation which can be used mixed with water. It contains, inter alia, glycerol, citric acid and hyaluronic acid, but does not contain ethanol.

It is known (WO 2005067944 A1) that hyaluronic acid, a glycosaminoglycan, is suitable for the treatment of inflammatory skin or mucosal diseases, in particular inflammatory atopic skin diseases.

WO 2008072905 A1 describes compositions for prevention and/or treatment of atopic dermatitis, comprising hyaluronic acid and/or a hyaluronic acid salt and resveratrol.

US 20190209606 A1 describes in situ gelling compositions comprising an anti-inflammatory polysaccharide and a gelling polymer, wherein the composition is a liquid prior to administration to a patient, but converts to a gel upon administration to the patient. Hyaluronic acid (also called hyaluronan, abbreviated as HA, CAS: 9004-61-9) is a glycosaminoglycan that is an important constituent of connective tissue. HA is characterized by a disaccharide repeating unit according to the following structure:

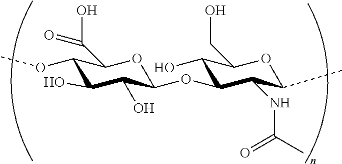

HA is a linear, acidic polysaccharide consisting of a multiplicity of alternating disaccharide units of 1,3-glycosidically linked N-acetyl-β-D-glucosamine (GlcNAc) and β-D-glucuronic acid (GlcA) molecules. Each of the disaccharidic base units is linked to the next by a β(1-4) bond. The number of base units can reach more than 10 000 with a molar mass of 4 million daltons. Owing to its hydrophilic acid groups and the hydroxyl groups, HA has the ability to form a multiplicity of hydrogen bonds which allow hydration and therefore make it homogeneously miscible with water. In aqueous solution, HA is capable of binding more and more water with widening of the average inter-chain distance, since the negative charges cause repulsion of the chain sections that lie close together. Under physiological conditions, the carboxyl groups of HA are practically completely dissociated and represent a polyanionic compound. The term hyaluronic acid is generally used irrespective of whether HA itself or the salts are meant. Owing to the viscoelastic properties and its biocompatibility, HA is of therapeutic importance as a solution for injection in ophthalmology, orthopedics and cosmetic surgery.

Hyaluronic acid is a natural constituent of the skin. In cosmetic products, use is made of two forms of hyaluronic acid, a short-chain and a long-chain form. The short-chain hyaluronic acid penetrates the upper layer of the skin and influences the inner moisture balance. By contrast, the long-chain hyaluronic acid is intended to exhibit a plumping-up effect on the skin. It effectively binds moisture in the upper layer of the skin. It has a hygroscopic effect because it releases its hydration shell only gradually and thus acts on the skin over a long period. HA has the further functions of supplying connective tissue and skin with moisture and nutrients.

The sodium salt of hyaluronic acid is, inter alia, also used as a moisturizer for the production of cosmetic products (Römpp online Lexicon, version 2.5, 2004).

Hyaluronic acid is commercially available in the crosslinked state (for example Hylaform®, a crosslinked hyaluronic acid from Biomatrix, NJ, USA; for preparation cf. also U.S. Pat. Nos. 4,713,448, U.S. Pat. No. 4,605,691, APC® from Fidia, Incert® from Anika Therapeutics, Intergel® from LifeCore or Restylane® from Q-Med).

Besides treatment of skin diseases such as atopic dermatitis, skin care is an important prophylactic approach. For the care of skin, a multitude of cosmetic preparations, usually in the form of creams and lotions, i.e. as an emulsion, are nowadays available to consumers. Products which temporarily or permanently delay or eliminate signs of skin aging, adverse environmental influences and specifically skin diseases are of ever-increasing importance. In addition to water for skin moisturization and oils and lipids for skin refatting, such skin care products comprise a multitude of active ingredients, excipients and additives.

There are countless studies on active ingredients usable for countering skin conditions, eczemas and atopic dermatitis. However, these active ingredients are often associated with adverse effects, such as corticoids, and so the benefit of lessening diseases is accompanied by the drawback of harm in other areas.

Furthermore, numerous clinical studies in the context of atopic dermatitis indicate that just the use of moisturizing agents alone can bring about a significant therapeutic effect that in some cases can be greater than that of added active ingredients (Vehicles for atopic dermatitis therapies: more than just a placebo, Journal of Dermatological Treatment, DOI: 10.1080/09546634.2020.1789050). However, these moisturizing agents are distinguished by the content of emollients and occlusives that are essential for an effect.

Furthermore, many of the active ingredients cannot be readily stably and storably incorporated in preparations for topical application.

It is desirable to provide a topically applicable preparation that is stable and storable and advantageously dispenses with active ingredients that have adverse effects.

It is desirable to provide a preparation that can be used for skin care and for treatment of inflammatory skin conditions and indications, such as acne, rosacea and/or atopic dermatitis.

It is also desirable to provide a preparation that is suitable for wound healing and for care of hypersensitive skin.

The invention is a topically applicable aqueous preparation, in particular cosmetic or dermatological preparation, comprising a substance combination of hyaluronic acid and/or hyaluronic acid salt, one or more fruit acids, one or more skin moisturizing agents and one or more alcohols.

Unlike known preparations in the treatment of atopic dermatitis, the preparation does not comprise emollients.

In particular, the present invention relates to the nontherapeutic use, in particular cosmetic use, of the preparation for promotion of the development, stabilization or shifting of an existing skin microbiome.

The nontherapeutic use of the preparation according to the invention for treatment of inflammatory skin conditions and/or indications, in particular acne, rosacea and atopic dermatitis, is advantageous.

The preparation according to the invention can likewise be advantageously used for skin care, in particular for care, prophylaxis and treatment of skin damaged by atopic dermatitis. The preparation according to the invention can likewise advantageously be used in treating atopic dermatitis and/or allergic contact dermatitis.

The preparation according to the invention can likewise advantageously be used in wound healing and for care of hypersensitive skin.

Although the individual substances of the combination according to the invention are known per se to a person skilled in the art in the field of cosmetics, there has been no prior disclosure or achievement of incorporating the combination of hyaluronic acid, fruit acids, one or more skin moisturizing agents and one or more alcohols in an effective form into cosmetic preparations, in particular hydrogels, and of being able to use it effectively against atopic dermatitis.

It is known that a superinfection with S. aureus can lead to skin inflammation.

It is likewise known that a healthy skin microbiome can minimize said skin inflammation.

The problem addressed is therefore that of providing a preparation for topical application that leads in particular to a reduction in S. aureus and simultaneous balancing of the skin microbiome, thereby resulting in an improvement in, in particular, atopic dermatitis.

Besides water, the preparations according to the invention comprise a combination of substances composed of hyaluronic acid and/or hyaluronic acid salt, one or more fruit acids, one or more skin moisturizing agents and one or more alcohols.

Hyaluronic acid and/or hyaluronic acid salt is preferably used in a proportion in the range from 0.05% to 3% by weight, in particular in the range from 0.5% to 1.5% by weight, preferably in the range from 0.7% to 1.2% by weight, based on the total mass of the preparation.

Preferably used according to the invention is sodium hyaluronate, CAS: 9067-32-7, poly(β-glucuronic acid-[1→3]-β-N-acetylglucosamine-[1→4]) alternating, (C₁₄H₂₁NaNO₁₁)ₙ, 1800-2500 kDa.

One or more fruit acids are preferably used in a proportion in the range from 0.005% to 2% by weight, in particular in the range from 0.01% to 1% by weight, preferably in the range from 0.02% to 0.5% by weight, based on the total mass of the preparation.

Advantageously used as fruit acids are the alpha-hydroxy acids (AHAs) and in particular glycolic acid, lactic acid, malic acid, citric acid and/or tartaric acid.

Preference is given to using citric acid.

According to the invention, the salts of the fruit acids, such as sodium citrate, which is known as a food additive, are not a fruit acid.

One or more skin moisturizing agents are preferably used in a proportion in the range from 0.1% to 15% by weight, in particular in the range from 0.5% to 10% by weight, preferably in the range from 1% to 7% by weight, based on the total mass of the preparation.

Moisturizing agents, also called moisturizers, refer to substances or compositions which endow cosmetic or dermatological preparations with the property of reducing moisture release from the stratum corneum (also called transepidermal water loss (TEWL)) and/or positively influencing hydration of the stratum corneum after said preparations have been applied to or spread on the surface of the skin.

Advantageous moisturizers in the context of the present invention are, for example, glycerol, caprylyl glycol, butylene glycol, propylene carbonate, urea, propylene glycol, hexanediols, aloe vera. It is particularly advantageous to select the combination of glycerol and caprylyl glycol as the skin moisturizing agents according to the invention.

One or more alcohols are preferably used in a proportion in the range from 0.05% to 15% by weight, in particular in the range from 1% to 10% by weight, preferably in the range from 2% to 8% by weight, based on the total mass of the preparation.

According to the invention, alcohols are understood to mean n-alkanols with n in the range from 1 to 4.

Ethanol is advantageously used as alcohol.

Advantageously, sodium hydrogencarbonate is added to the preparation according to the invention, in particular in a proportion by weight in the range from 0.5% to 10% by weight, in particular in the range from 1% to 9% by weight, based on the total mass of the preparation.

According to the invention, at least one representative is present from each group of substances and is different from the other representatives, meaning that two different substances are present even if one representative should have twin properties. For example, hyaluronic acid is also a skin moisturizing agent. Therefore, according to the invention, another skin moisturizing agent is present in addition to hyaluronic acid.

According to the invention, emollients are dispensed with.

Emollients are agents for making the skin soft and supple (for softening the skin). Emollients are substances that reduce skin roughness and thus make the skin appear softer and smoother. They act by “filling” the spaces between desquamating corneocytes, by increasing cohesion between corneocytes and by “smoothing” the raised edges of individual corneocytes.

Examples of commonly used emollients are oils and fats, such as lanolin, mineral oil and Vaseline. Some emollients are, for example, vegetable oils, synthetic and semisynthetic oils, such as cetyl ethylhexanoate, isopropyl myristate, ethylhexyl palmitate, caprylic/capric triglyceride, and some silicones.

According to the invention, these emollients are dispensed with, and so the proportion thereof in the preparation is not more than 0.1% by weight.

The combination of substances according to the invention and the individual constituents thereof are therefore not understood as meaning emollients.

For atopic dermatitis, the literature recommends that it be treated generally, in accordance with the symptoms, with an adapted and graduated therapy. An essential component of nondrug therapy is daily treatment with moisturizing agents, known as moisturizers, emollients, i.e., lipophilic ingredients for smoothing and softening the skin, occlusives, i.e., substances for preventing water loss and providing protection against external irritation, and humectants, i.e., substances for moisturizing the skin by active binding and retention of water (Nicol N.H., Rippke F., Weber T.M., Hebert A.A., The Journal for Nurse Practitioners 2021, 17, 920-925).

However, according to the invention, the use of emollients is dispensed with, and only the combination of substances according to the invention suffices for performing the stated functions. According to the invention, what is meant by dispensing with emollients is that the proportion of emollients in the preparation is less than 0.1% by weight, and so any negligible proportions, for example due to impurities or entrained materials with emollients, are still in accordance with the invention.

Preferably, the proportion of emollients is less than 0.01% by weight, in particular less than 0.001% by weight, advantageously 0% by weight, based on the total mass of the preparation. Fats, oils and waxes, if not already covered by emollients, are also dispensed with.

Lipids, such as fats, oils and waxes, are therefore present in the preparation at not more than less than 0.1% by weight, and so any negligible impurities or entrained materials with lipids are still in accordance with the invention.

Preferably, the proportion of lipids is less than 0.01% by weight, in particular less than 0.001% by weight, advantageously 0% by weight, based on the total mass of the preparation.

The combination of substances according to the invention and the individual constituents thereof are therefore not understood as meaning lipids.

According to the invention, emulsifiers can advantageously likewise be dispensed with, and so the proportion thereof in the preparation is preferably less than 0.1% by weight.

Preferably, the proportion of emulsifiers is less than 0.01% by weight, in particular less than 0.001% by weight, advantageously 0% by weight, based on the total mass of the preparation.

The preparation according to the invention is topically applicable, i.e., it is applied to the skin and not to the mucosa, oral mucosa or nasal mucosa.

Cosmetic preparations are used in various forms, such as cream, paste, lotion or gel. The preparation according to the invention is advantageously provided as a hydrogel.

It is an essential object of the present invention to achieve relief of atopic dermatitis (AD) with acute to subacute eczema, i.e., acute inflammation. Surprisingly, the hydrophilic preparation according to the invention is especially distinguished by water-binding constituents (HA, glycerol) and readily evaporating constituents (water, ethanol) which can contribute to cooling acute inflammation.

Furthermore, it is preferred according to the invention if the preparations according to the invention are stored at cool temperatures, for example in a refrigerator, ideally at 4° C., and are thus applied chilled in order to additionally support the cooling effect.

The invention is therefore also a method for applying the preparation according to the invention to skin, characterized by a first step of providing the preparation and/or preparing it by mixing the ingredients, a second step of adjusting the temperature of the preparation (cooling the preparation) to between 1° C. and 10° C., preferably between 2° C. and 8° C. and especially preferably between 3° C. and 6° C., and a third step of transferring the temperature-adjusted preparation to human skin. Use of the method further improved the invention.

The preparations according to the invention meet all the stated objects.

Surprisingly, the preparations according to the invention moreover show that the healthy skin microbiome is promoted and stabilized.

The preparations according to the invention can thus be used to promote the development, stabilization or shifting of an existing skin microbiome.

Tests were carried out in relation to this, in which preparations according to the invention show the growth of S. epidermidis, as a representative bacterial species of a healthy skin microbiome, on the skin and at the same time selectively damage only S. aureus.

These and further tests demonstrate that the preparations according to the invention promote a healthy skin microbiome by supporting the growth of S. epidermidis, as a representative bacterial species, and at the same time selectively damaging disruptive species, such as S. aureus.

The preparations according to the invention therefore promote the development, stabilization and/or shifting of an existing skin microbiome toward a healthy state.

Thus the preparations according to the invention are also suitable for cosmetic skin care, i.e., nontherapeutic skin care.

Preference is thus also given to the nontherapeutic use of the preparations according to the invention for treatment of inflammatory skin conditions and/or indications, in particular acne, rosacea, atopic dermatitis, and additionally for care, prophylaxis and treatment of atopic dermatitis and for wound healing or care of hypersensitive skin.

In the tests, the preparation listed in Example 1 was used twice daily for a period of three weeks.

The preparation used was:

### EXAMPLE 1 (% BY WEIGHT)

0.9% sodium hyaluronate
5% glycerol
0.11% caprylyl glycol
8% ethanol
0.04% citric acid
ad 100% water
pH = 5.8 − 6.0

The subjects participating in the test had the following criteria:
skin type (according to the Fitzpatrick scale) between I and IV
male and female volunteers diagnosed with atopic dermatitis which, however, does not currently require drug treatment
diagnostic criteria for atopic dermatitis: a local SCORAD of at least 6, preferably >10, at least 2 comparable lesions on comparable areas of the body, with a minimum size of 2×2cm, preferably on the inner forearms, the crook of the arm or the back of the knee.

### Procedure

A cohort of 16 subjects was invited to apply, for a period of 3 weeks, 2×0.6 ml of preparation (Example 1) per day to a 100 cm² area of skin which had to contain an at least 2×2 cm lesion typical of atopic dermatitis at the centre. The distance between the lesion and the edge of the area should be at least 2 cm.
The preparation was spread in a circular motion in the area for a period of 30 seconds
After the preparation had been applied, the treated area was air-dried for at least 15 minutes until liquid was no longer visible
The use amount should not be exceeded.

The evaluation of the test was divided into:
Reporting of adverse effects after each application by a dermatologist. (Photographic) documentation in the event of an adverse reaction.
Objective dermatological evaluation by a dermatologist and determination of the local SCORAD (SCORing Atopic Dermatitis) index at the following times:

$$\begin{matrix}- T 0 = basline \\ - T 2 = day 2 \\ - T 3 = day 5 \\ - T 4 = day 8 \\ - T 5 = day 9 \\ - T 6 = day 12 \\ - T8 = day 22\end{matrix}$$

SCORAD (SCORing Atopic Dermatitis) is a clinical assessment system used for determining the severity of atopic dermatitis in patients as objectively as possible. The SCORAD system was developed in 1993 by the European Task Force on Atopic Dermatitis.

The local SCORAD was determined in accordance with Angelova-Fischer et al. by
Clinical photography
Self-assessment questionnaire: The following skin parameters were tested on a 10-point scale:
   1) Skin moisturization
   2) Skin smoothness
   3) Overall skin condition
   4) Skin redness
   5) Sense of wellbeing
   6) Tight skin feel
   7) Itchy skin feel
   8) Burning skin feel
   9) Skin reactivity sensitivity

(Angelova-Fischer I., Rippke F., Richter D., Filbry A., Arrowitz C., Weber T., Fischer T.W., Zilllikens. Acta Dermato-Venereologica 2018. 98. 517-523).

TABLE 1
Scale for subjects to assess the various skin parameters

| | Criteria | Assessment | Score of 1 | Score of 10 |
|---|---|---|---|---|
| 1 | Skin | . . . of the dryness of the lesioned skin | Very dry & | Very highly |
| | moisturization | in the test region. Viewing of the | scaly | moisturized, no |
| | (visual) | skin with lesions from different | | signs of dryness |
| | | angles and noting of signs of scaling | | and/or scales |
| | | and dryness. | | |
| 2 | Skin smoothness | . . . of the roughness of the lesioned | Very rough | Very soft |
| | (visual) | skin in the test region. Observation | | |
| | | of the skin lesion and noting of | | |
| | | wrinkles and skin irregularities in | | |
| | | the surface structure. | | |
| 3 | Overall skin | . . . of the total skin score for the | Very poor | Very good skin |
| | condition | lesioned skin. Consideration of signs | skin | condition |
| | (visual) | of dryness, scaling and redness. | condition | |
| 4 | Skin redness | . . . of redness. Observation of the | Very severe | No obvious |
| | (visual) | skin lesion from different angles and | redness | redness (apart |
| | | noting of red spots and red regions. | | from rosy skin |
| | | | | tone) |
| 5 | Sense of | . . . of discomfort in the skin lesion. | Strong, | No unpleasant |
| | wellbeing as | Noting of sensation of tightness, | unpleasant | sensation at all |
| | regards skin | itching, tingling and burning. | sensation | |
| | (feel) | | | |
| 6 | Tight skin feel | . . . of sensation of tightness in the | Strong, | No sensation of |
| | (feel) | lesioned skin. | frequent | tightness at all |
| | | | sensation of | |
| | | | tightness | |
| 7 | Itchy skin feel | . . . of itching of the lesioned skin. | Severe, | No itching at all |
| | (feel) | | frequent | |
| | | | itching | |
| 8 | Burning skin | . . . of burning sensation in the | Strong, | No burning |
| | feel | damaged skin. | frequent | sensation at all |
| | (feel) | | burning | |
| | | | sensation | |
| 9 | Skin reactivity | . . . of the reactivity and sensitivity of | Frequent, | Very tolerant, |
| | sensitivity (feel) | the skin with lesions. Assessment of | extremely | highly |
| | | whether and how often/severely the | reactive, | insensitive |
| | | skin has reacted to daily stresses | sensitive | |
| | | such as extreme weather, wind, dry | | |
| | | heating air, surfactants or other | | |
| | | unpleasant materials such as wool. | | |

During the study, the preparation did not cause any adverse effects. The subjects generally had very positive comments for the formula. The preparation was found by the subjects to exhibit good and rapid absorption, a low viscosity, a more pleasing texture (compared to creams containing emollients) and a refreshing character. As a result, some subjects used the formula on areas of the body other than the ones defined.

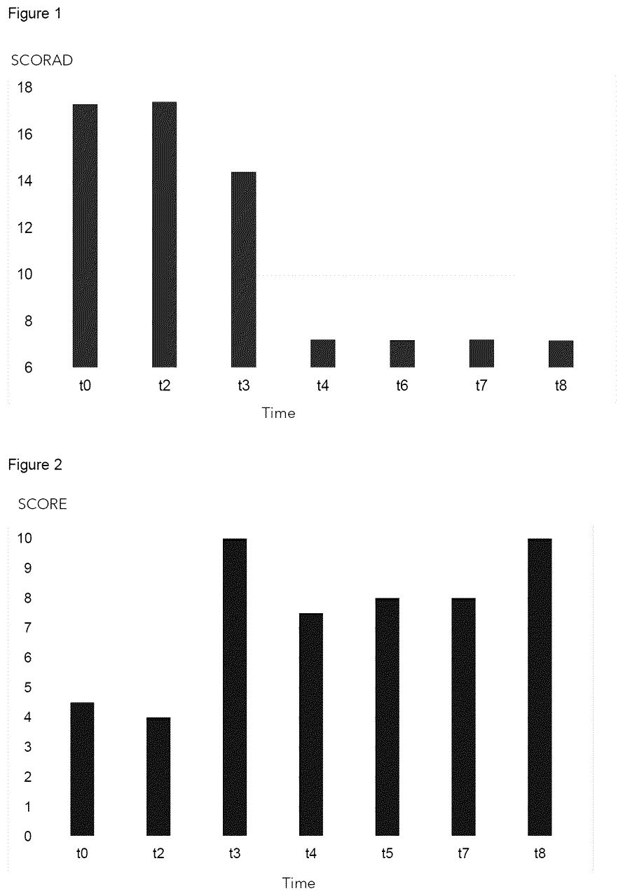

Remarkably, the preparation according to the invention was able to significantly reduce the local SCORAD after just 5 days (t3), thus generally improving the local SCORAD. In 7 of these 13 subjects, a reduction to 0 was achieved in 22 days (18), which means freedom from symptoms. On average, an improvement in local SCORAD from 17.3 to 7.2 (median) was determined, which corresponds to a 58.5% improvement in 3 weeks. These results are surprising because the preparation does not contain any typical active ingredients (against inflammation or itching) or any emollients that would typically be used to treat such acute phenotypes of atopy. FIG. 1 and the table below show the results of the clinical/dermatological assessment (median local SCORAD) of the preparation-treated areas over time.

FIG. 1 shows the local SCORAD (median) over time (t0-t8)/(SCORAD 0 to 20)

Table 2 shows the clinical/dermatological assessment, the local SCORAD, and the median of the differences in relation to the starting value (t0)

TABLE 2
Changes compared to starting value (tᵢ − t₀)

| Clinical/dermatological | | | | | | |
|---|---|---|---|---|---|---|
| assessment | t2 | t3 | t4 | t5 | t7 | t8 |
| Local Scorad | 0.0 | −3.0* | −7.0* | −8.5* | −9.5* | −10.1* |

*Significant reduction in local Scorad compared to the initial state, two-sided Wilcoxon signed rank test with significance level α = 0.05

Subjective assessment by the subjects:

FIGS. 2-9 and Table 3 show the results from surveying the subjects on the subjective parameters: skin reactivity sensitivity, itchy skin feel, tight skin feel, general sense of wellbeing, skin redness, overall skin condition, skin smoothness and skin moisturization. These were assessed on a 10-point scale (score of 1 (poor)-10 (good)). Generally and surprisingly, each parameter that started at t0<10 was improved. The majority of the further parameters were assessed on average with a moderately poor starting value of 4 at starting time t0. After 5 days (t3), a statistically significant improvement was observed for all parameters.

FIG. 2 shows skin reactivity sensitivity.

A significant increase in score compared to the initial state (=improvement) is found, two-sided Wilcoxon signed rank test with significance level α=0.05.

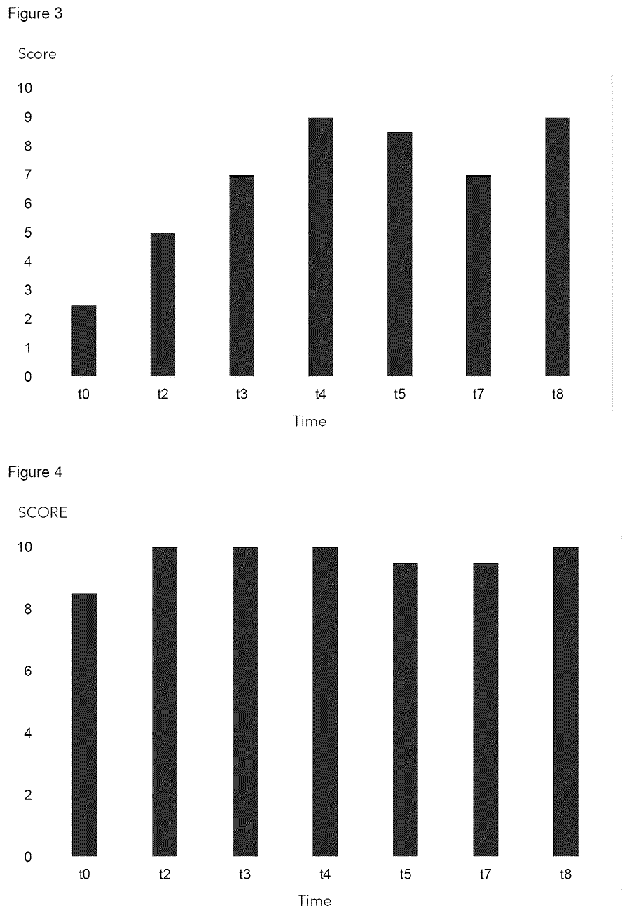

FIG. 3 shows itchy skin feel and a significant increase in score compared to the initial state (=improvement)*.

FIG. 4 shows the assessment of tight skin feel. Here too, there is a significant increase in score compared to the initial state (=improvement)*.

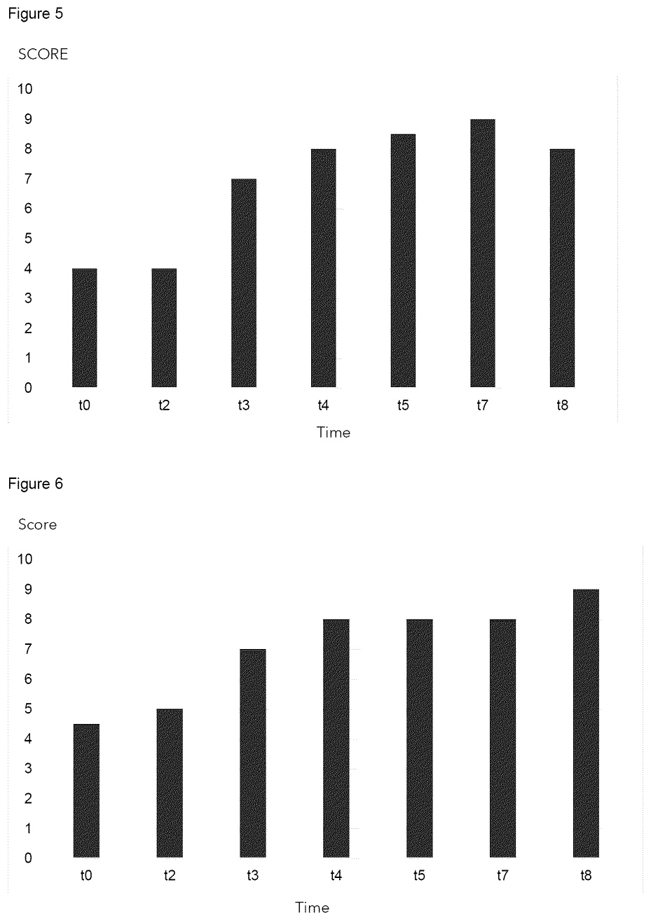

FIG. 5 shows sense of wellbeing. The result is a significant increase in score compared to the initial state (=improvement)*.

FIG. 6 shows skin redness, which likewise shows a significant increase in score compared to the initial state (=improvement)*.

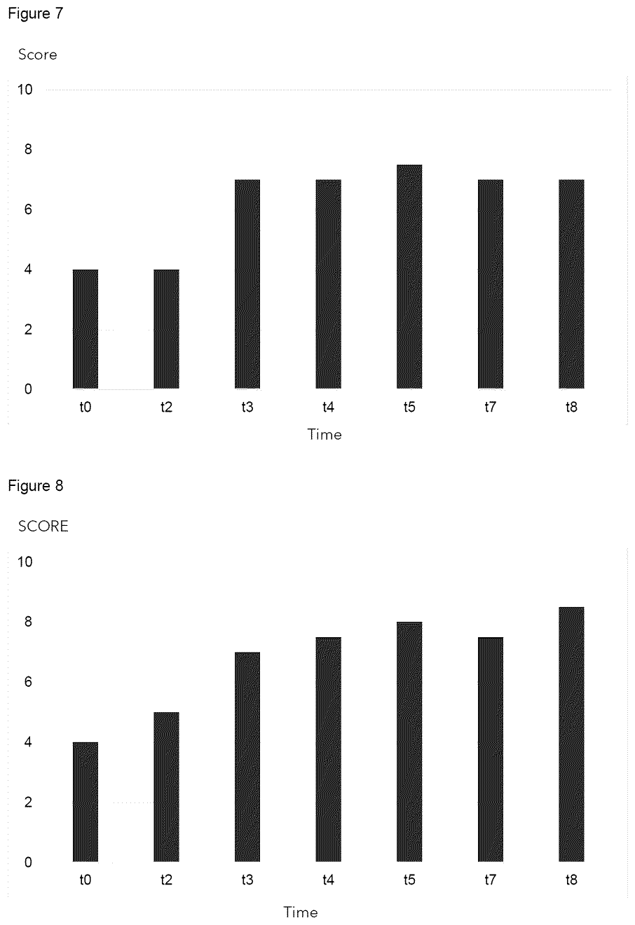

FIG. 7 shows overall skin condition with likewise a significant increase in score compared to the initial condition (=improvement)*.

FIG. 8 shows skin smoothness. A significant increase in score compared to the initial state (=improvement) is found*.

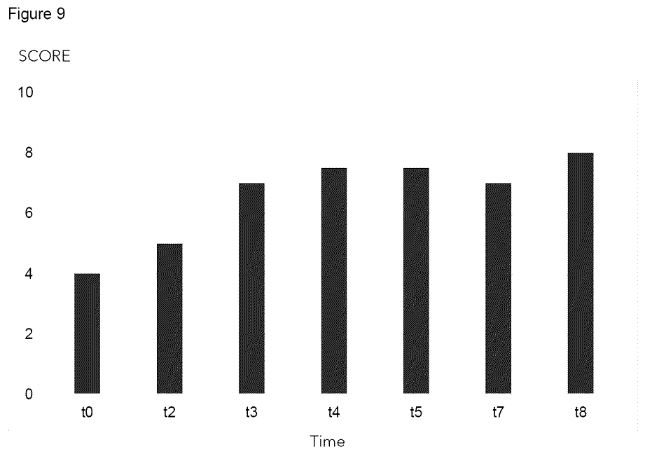

FIG. 9 shows skin moisturization and a significant increase in score compared to the initial state (=improvement)*.

TABLE 3
Subjective assessment by subjects, scores, mean of the
differences in relation to the starting value (t0)
Changes compared to starting value (tᵢ − t₀)

| Subjective assessment by | | | | | | |
|---|---|---|---|---|---|---|
| the subjects | t2 | t3 | t4 | t5 | t7 | t8 |
| Skin moisturization | 0.9* | 2.7* | 3.1* | 3.2* | 2.9* | 3.2* |
| Skin smoothness | 0.3 | 2.0* | 2.4* | 2.6* | 2.6* | 2.9* |
| Overall skin condition | 0.2 | 2.3* | 2.9* | 2.7* | 2.3* | 2.4* |
| Skin redness | 0.8* | 2.5* | 3.4* | 2.9* | 2.8* | 3.3* |
| General sense of wellbeing | 0.2 | 2.2* | 3.3* | 3.8* | 3.3* | 2.7* |
| Tight skin feel | 1.5 | 1.7 | 0.9 | 1.2 | 1.5 | 1.1 |
| Itchy skin feel | 1.6 | 3.9* | 5.3* | 4.8* | 3.3* | 3.9* |
| Burning skin feel | 2.1* | 2.3* | 2.3* | 2.2* | 2.3* | 1.9* |
| Skin reactivity sensitivity | 0.3 | 4.0* | 2.4* | 2.6* | 2.8* | 3.4* |

*Significant increase in score compared to the initial state (=improvement), two-sided Wilcoxon signed rank test with significance level α = 0.05.

Cosmetic preparations and their specific formulations can bring about cumulative irritation and therefore cause irritative contact dermatitis in consumers. The most important pathological mechanisms of irritation include disruption of the skin barrier, which can lead to a visible reaction, erythema. The irritation potential of the preparations according to the invention was tested by carrying out blinded and comparative epicutaneous patch studies for determination of in vivo skin compatibility in healthy subjects.

What was tested was the preparation according to the invention as per Table 4 below:

| INCI | % by weight |
|---|---|
| Sodium Hyaluronate | 0.9 |
| Glycyrrhiza Inflata Root Extract | 0.025 |
| Hydroxyacetophenone | 0.23 |
| Glycine | 0.1 |
| Vitis Vinifera Seed Oil | 0.05 |
| Glycerin + Aqua | 10 |
| Aqua + Sodium PCA | 0.1 |
| Citric Acid | 0.06 |
| Alcohol Denat. + Aqua | 8 |
| Gellan Gum | 0.25 |
| Aqua | 80.285 |

The results of clinical testing by means of a Cumulative Irritation Patch Test (REP) to test skin compatibility on the back under controlled conditions showed that the formulation according to the invention has no irritation potential and has good skin compatibility.

In a further study, the skin compatibility of a face and body gel according to the invention was determined in vivo in subjects with sensitive skin and mild atopic dermatitis.

What was tested was a hydrogel having the composition as per Table 5:

| | |
|---|---|
| Sodium Hyaluronate | 0.9 |
| Glycyrrhiza Inflata Root Extract | 0.025 |
| Hydroxyacetophenone | 0.23 |
| Glycine | 0.1 |
| Vitis Vinifera Seed Oil | 0.05 |
| Glycerin + Aqua | 10 |
| Aqua + Sodium PCA | 0.1 |
| Citric Acid | 0.06 |
| Alcohol Denat. + Aqua | 8 |
| Aqua | 80.535 |

The test product was used by application twice daily to the body and the face according to the physician's instructions and the participant's information sheet for a total period of two weeks. The use of other skin care products during the study was not permitted.

The subjects consisted of 43 volunteers (14 men and 29 women) aged from 23 to 71 years, with 41 participants having dry skin. All 43 participants were diagnosed with at least one current dermatological disease (100%, atopic dermatitis (n=41, 95.3%), xerosis (n=32, 74.4%)).

The skin was examined at the start of the study and at the end of the two-week study. The assessment parameters were visually examined by a physician: dryness, scaling, erythema, itchy skin feel, tight skin feel/tight skin and burning skin feel.

The symptoms were classified on a 5-point scale (none to very severe). At the end of the study, the physicians also assessed the skin condition for comparison with the starting value and the compatibility/tolerability of the test product.

In summary, the study showed that, under the conditions used in said study, the hydrogel according to the invention proved to have very good skin compatibility in the participants with sensitive skin and atopic dermatitis. The hydrogel had a very positive effect on the overall skin condition of the body and face.

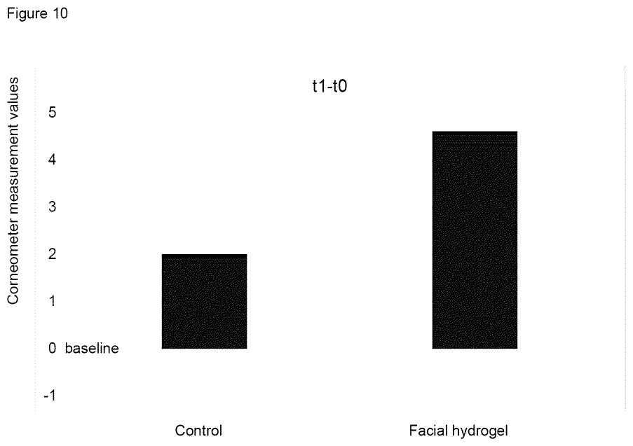

In a further use study, the effectiveness of a facial care product according to the invention was tested. The study was designed to determine positive effects on the skin condition at the treated spots in comparison with the baseline and with an untreated control under standard use conditions, with examination of particular aspects such as signs of dryness or hydration. The in vivo design of the study ran for 2 assessments over a period of 1 day per subject: baseline (t0) and 24 hours after single use (t1) of the test product (FIG. 10).

Treatment: Single application of the test products to the inner sides of the forearms by the subject, one test spot remained untreated as control.

Study population: 35 female subjects aged from 19 to 65 years who were qualified for the study. Participation by demonstration of healthy skin on the basis of a self-assessment and also by participation in a preconditioning period.

The following methods were used to assess the effects of the product on the skin:

Changes in condition:
a.) Corneometer CM 825 measurements were carried out in order to ascertain the moisturizing care on the inner forearms to be assessed
b.) A self-assessment by the subjects in order to the condition and/or appearance on the inner forearms to be assessed

The results of this controlled study showed the skin care effectiveness of the preparation according to the invention as per Table 6.

TABLE 6

| INCI | % by weight |
|---|---|
| Sodium Hyaluronate | 0.9 |
| Glycyrrhiza Inflata Root Extract | 0.025 |
| Hydroxyacetophenone | 0.23 |
| Glycerin + Aqua | 10 |
| Citric Acid | 0.04 |
| Alcohol Denat. + Aqua | 8 |
| Aqua | 80.805 |

Various improvements in skin condition were found and they were already observed after single use.

The Corneometer CM 825 values increased significantly within 24 hours after single use. Moreover, an improved skin appearance and a significant increase in skin moisture were observed by the self-assessment of the subjects 24 hours after single use.

In summary, the study supports the assertion that preparations according to the invention moisturize the skin, which is demonstrated in particular by the increased skin moisture by Corneometer measurements (FIG. 10).

An exploratory study was carried out to investigate the anti-irritant, anti-redness activity of the preparations according to the invention on the forearm in comparison with an untreated control and a shaved untreated control.

Preparations according to the invention were found to lead to an improvement in skin condition following skin irritation (in this case: shaving).

Female subjects with healthy skin on the palms of their hands were enrolled in this study. Irritation was induced in a standardized manner by repeated stroking with a razor blade on the test surface. The effect of the test product was assessed by subjective assessment of skin sensation immediately after use and after three consecutive days after shaving and product use. In addition, skin redness was assessed by chroma meter measurements at the end of the study.

The test product was applied twice daily to a shaved test spot during these three days: immediately after shaving and in the evening. Another shaved test spot was left untreated and used as control.

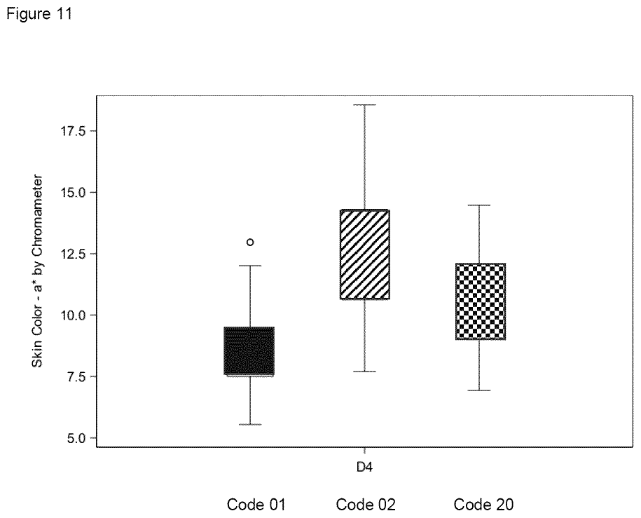

The result of this study was that the irritated skin (skin redness induced by shaving) was distinctly reduced after 3 days of treatment with the tested product in comparison with untreated and shaved skin. This was confirmed by chroma meter measurements and by self-assessment by the subjects. Skin calming was significantly improved after 3 days of treatment with the test product in comparison with untreated, shaved skin. The study confirms that preparations according to the invention have an anti-irritant/irritant effect, bring relief, reverse anti-redness and have a calming effect. Furthermore, the preparations according to the invention are even suitable for irritated, sensitive skin on the basis of the shaving method. Skin redness a* was tested with a chroma meter after 3 days (on day 4, d4) for an untreated skin area (code 01), a shaved and untreated skin area (code 02) and a shaved and treated skin area (code 20) (FIG. 11).

Treatment was carried out with a test product, a hydrogel according to the invention, according to Table 7:

| INCI | % by weight |
|---|---|
| Sodium Hyaluronate | 0.9 |
| Glycyrrhiza Inflata Root Extract | 0.025 |
| Hydroxyacetophenone | 0.23 |
| Glycine | 0.1 |
| Vitis Vinifera Seed Oil | 0.05 |
| Glycerin + Aqua | 10 |
| Aqua + Sodium PCA | 0.1 |
| Citric Acid | 0.06 |
| Alcohol Denat. + Aqua | 8 |
| Aqua | 80.535 |

Advantageously according to the invention, the combination of substances composed of hyaluronic acid and/or hyaluronic acid salt, one or more fruit acids, one or more skin moisturizing agents and one or more alcohols is provided in a preparation based on a hydrogel.

Hydrogel refers to gels having a high water content. They consist mainly of hydrophilic polymers which can swell considerably in water while largely maintaining their shape. In the hydrogel according to the invention, hyaluronic acid is the water-binding substance. Emulsifiers can advantageously also be dispensed with.

As preferred hydrogel, emulsifiers are likewise advantageously not used, and so the proportion thereof in the preparation is preferably less than 0.1% by weight. Preferably, the proportion of emulsifiers is less than 0.01% by weight, in particular less than 0.001% by weight, advantageously 0% by weight, based on the total mass of the preparation. Thus the preparations according to the invention are preferably not emulsions.

A preferred preparation according to the invention consists only of hyaluronic acid and/or hyaluronic acid salt, one or more fruit acids, in particular citric acid, one or more skin moisturizing agents, in particular glycerol and/or caprylyl glycol, and one or more alcohols, in particular ethanol, and water, as shown in particular in Table 8 below.

| INCI | % by weight |
|---|---|
| Sodium Hyaluronate | 0.9 |
| Glycerin + Aqua | 5 |
| Caprylyl Glycol + Aqua | 0.35 |
| Citric Acid | 0.01 |
| Alcohol Denat. + Aqua | 8 |
| Aqua | 85.74 |

According to the invention, the preparations are used to promote the development or stabilization of a healthy skin microbiome.

Not only can the preparations according to the invention improve skin care, but they also provide in particular a means for treatment of atopic dermatitis.

The preparations according to the invention can be used in various application forms. Preferred application forms are gels, sprays, serums, soaking medium for soaked patches, wipes or masks.

The demanding cosmetic compositions according to the invention can optionally comprise further customary excipients and additives, such as texturing agents, fillers, perfume, dyes, active ingredients, vitamins, proteins, light stabilizers, stabilizers, insect repellents, salts, EDTA, antimicrobially, proteolytically or keratolytically active substances, etc., provided that they are not excluded according to the invention.

Preferably according to the invention, one or more active ingredients from the following groups are added to the preparations in order to be able to ensure broadest possible use forms and individual care and treatments.

What can advantageously be added to the preparations or application forms according to the invention are active ingredients such as, preferably, antibacterial active ingredients, antifungal active ingredients, sebum-reducing active ingredients, itching-relieving active ingredients, anti-inflammatory, pro-healing, analgesic or antimicrobial active ingredients, such as dexpanthenol, or natural additives, natural products, such as honey, chamomile or aloe vera. Further possible additional active ingredients are vitamins such as vitamin A, C or E, growth factors such as PDGF, sugars or polysaccharides such as glucose, minerals such as zinc, amino acid and derivatives thereof such as arginine or creatine.

Also preferred according to the invention is the addition of cell membrane-addressing active ingredients, such as quats, octenidines or decanediol.

Especially natural products such as cyclic and noncyclic peptides of ribosomal and nonribosomal (NRPS-derived) origin and cyclic and noncyclic polyketides (PKS-derived) can advantageously be added according to the invention.

Further preferred active ingredients are enzymes, especially cell-wall hydrolytic enzymes such as endolysins, lysozyme and chitinases in this case.

Particularly preferred is the addition of one or more active ingredients selected from the group consisting of hydroxyacetophenone, salicylic acid, Glycyrrhiza inflata root extract, licochalcone A, Q10 and/or thiamidol, preferably hydroxyacetophenone.

Advantageously added from the group of antibacterial active ingredients are quats, in particular PQ16 Quat Excellence, octenidines, decanediol, quaternary amines, benzalkonium chloride and/or benzethonium chloride.

Natural products such as cyclic and noncyclic peptides of ribosomal and nonribosomal (NRPS-derived) origin, cyclic and noncyclic polyketides (PKS-derived) and peptide/polyketide conjugates (NRPS/PKS-derived) can also advantageously be added to the preparations according to the invention.

The addition of enzymes, especially cell-wall hydrolytic enzymes such as endolysins, lysozyme and chitinases (fungal cell wall), is preferred.

From the group of antifungal active ingredients, climbazole or piroctone olamine is added in particular.

Further preferred additives can be selected from Ca, Mg, Al, and/or Zn salts.

Further preferred active ingredients can be selected from Magnolia, arctiin, Bioxilift, creatine, isoflavones, Laminaria, NAHP, phloridzin, vitamin C, lotus extract, Myriceline, white tea, AGR, glycyrrhetinic acid, Silymarin S, tocopheryl, carnitine, Garcinia cambogia, Guarana C22, butyloctanoic acid, dioic, ethylhexylglycerin, methyl phenylbutanol, polyglyceryl-2 caprate, Polysaf 5600 polymer, silver citrate, zinc citrate, Betaine, sea salt, taurine, SymSave H, DHA, rucinol, panthenol/dexpanthenol.

The proportion of one or more additionally added active ingredients can be below 1% by weight, preferably less than 0.5% by weight, based on the total mass of the preparation.

In addition, essential oils can also be added to the preparations. Menthol, camphor and derivatives thereof, but also other essential oils, reduce the stimulus threshold of neural cold receptors, thus causing a sensation of cold. These active ingredients can also be used in a preferred embodiment. According to the invention, essential oils do not in this case belong to the group of lipids or emollients.

1.-15. (canceled)

16. A aqueous preparation for topical application to skin, wherein the preparation comprises (i) hyaluronic acid and/or hyaluronic acid salt, (ii) one or more fruit acids, (iii) one or more skin moisturizing agents, and (iv) one or more alcohols, and wherein emollients and lipids are each present in a concentration of less than 0.1% by weight, based on a total weight of the preparation.

17. The preparation of claim 16, wherein the preparation comprises, based on a total weight of the preparation, from 0.05% to 3% by weight of (i) and/or from 0.005% to 2% by weight of (ii) and/or from 0.1% to 15% by weight of (iii) and/or from 0.05% to 15% by weight of (iv).

18. The preparation of claim 16, wherein the preparation comprises, based on a total weight of the preparation, from 0.05% to 3% by weight of (i), from 0.005% to 2% by weight of (ii), from 0.1% to 15% by weight of (iii), and from 0.05% to 15% by weight of (iv).

19. The preparation of claim 16, wherein the preparation comprises, based on a total weight of the preparation, from 0.5% to 1.5% by weight of (i), from 0.01% to 1% by weight of (ii), from 0.5% to 10% by weight of (iii), and from 1% to 10% by weight of (iv).

20. The preparation of claim 16, wherein the preparation comprises, based on a total weight of the preparation, from 0.7% to 1.2% by weight of (i), from 0.02% to 0.5% by weight of (ii), from 1% to 7% by weight of (iii), and from 2% to 8% by weight of (iv).

21. The preparation of claim 16, wherein (ii) comprises one or more alpha-hydroxy acids and/or (iii) comprises one or more of glycerol, caprylyl glycol, butylene glycol or propylene carbonate and/or (iv) comprises ethanol.

22. The preparation of claim 16, wherein (ii) comprises one or more alpha-hydroxy acids, (iii) comprises one or more of glycerol, caprylyl glycol, butylene glycol or propylene carbonate, and (iv) comprises ethanol.

23. The preparation of claim 22, wherein (ii) comprises one or more of glycolic acid, lactic acid, malic acid, citric acid or tartaric acid.

24. The preparation of claim 22, wherein (ii) comprises citric acid.

25. The preparation of claim 16, wherein the preparation further comprises one or more of hydroxyacetophenone, salicylic acid, Glycyrrhiza inflata root extract, licochalcone A, Q10 or thiamidol.

26. The preparation of claim 16, wherein the preparation further comprises sodium hydrogencarbonate.

27. The preparation of claim 26, wherein sodium hydrogencarbonate is present in a concentration of from 0.5% to 10% by weight, based on a total weight of the preparation.

28. The preparation of claim 16, wherein the preparation comprises less than 0.01% by weight of each of emollients, lipids, fats, oils and waxes, based on a total weight of the preparation.

29. The preparation of claim 16, wherein the preparation comprises less than 0.1% by weight of emulsifiers, based on a total weight of the preparation.

30. The preparation of claim 16, wherein the preparation consists of water and components (i), (ii), (iii) and (iv).

31. The preparation of claim 16, wherein the preparation is present as a hydrogel, spray, serum, soaking medium for patches, wipes or masks.

32. The preparation of claim 16, wherein the preparation is present as a hydrogel.

33. A method of treating an inflammatory skin condition, wherein the method comprises applying to skin affected by the inflammatory skin condition the preparation of claim 16 in an amount which is sufficient to treat the inflammatory skin condition.

34. The method of claim 33, wherein the inflammatory skin condition is one or more of acne, rosacea, or atopic dermatitis.

35. A method of applying the preparation of claim 16 to human skin, wherein the method comprises providing the preparation and/or preparing it by mixing its ingredients, adjusting a temperature of the preparation to from 1° C. to 10° C., and transferring the temperature-adjusted preparation to the human skin.
